(19) United States
(12) Patent Application Publication
(10) Pub. No.: US 2023/0233196 A1
(43) Pub. Date: Jul. 27, 2023

(54) LIVING TISSUE SAMPLING METHOD AND BIOPSY SUPPORT SYSTEM

(71) Applicant: OLYMPUS MEDICAL SYSTEMS CORP., Tokyo (JP)

(72) Inventors: Koichiro ITO, Tokyo (JP); Tetsuhide TAKEYAMA, Tokyo (JP); Takeshi SUGA, Tokyo (JP); Kazunari HANANO, Tokyo (JP); Keigo MATSUO, Tokyo (JP)

(73) Assignee: OLYMPUS MEDICAL SYSTEMS CORP., Tokyo (JP)

(21) Appl. No.: 18/118,897

(22) Filed: Mar. 8, 2023

Related U.S. Application Data
(63) Continuation of application No. PCT/JP2020/035579, filed on Sep. 18, 2020.

(51) Int. Cl. A61B 10/04; A61B 1/01; A61B 1/00; A61B 8/08; A61B 10/06; A61B 6/12; A61B 8/12; A61B 1/267

(52) U.S. Cl. CPC A61B 10/04; A61B 1/01; A61B 1/00009; A61B 1/2676; A61B 6/12; A61B 8/12; A61B 8/0841; A61B 10/06

(57) ABSTRACT

A living tissue sampling method includes inserting an endoscope inserted into a guide sheath into a body cavity of a subject, checking three-dimensional image information of a body cavity path against distal end position data of the endoscope, bringing distal ends of the endoscope and the guide sheath close to a focused region, removing the endoscope from the guide sheath while keeping the guide sheath stationed inside the body cavity, inserting a biopsy instrument into the guide sheath, and sampling living tissue of the focused region by the biopsy instrument.

### CROSS REFERENCE TO RELATED APPLICATION

This application is a continuation application of PCT/JP2020/035579 filed on Sep. 18, 2020, the entire contents of which are incorporated herein by this reference.

### BACKGROUND OF THE INVENTION

### 1. Field of the Invention

The present invention relates to a living tissue sampling method and a biopsy support system, and more particularly, to a living tissue sampling method and a biopsy support system that support a procedure of performing biopsy from target tissue in a tumor or the like by using an ultrasound tomographic image obtained by inserting an ultrasound probe into a lumen of a subject, such as a bronchus.

### 2. Description of the Related Art

Nowadays, a technique of performing observation/diagnosis in relation to a lumen such as a digestive tract, a bronchus, a biliopancreatic duct, a blood vessel or the like and a peripheral organ by using an endoscope that is capable of acquiring an optical image of a subject and a body cavity probe of an ultrasound endoscope or the like that is capable of acquiring a tomographic image of the subject is well known.

For example, as a guide system for performing observation/diagnosis of a lesion of a bronchus of a subject, Japanese Patent Application Laid-Open Publication No. 2014-204904 discloses a guide system for guiding an ultrasound probe that is inserted into a forceps channel of an endoscope, to enable access to surroundings of a lesion site.

An outline of a step of observing and performing biopsy (living tissue sampling) on a lesion site by the guide system disclosed in Japanese Patent Application Laid-Open Publication No. 2014-204904 is given below. First, a distal end portion of a bronchial endoscope inserted into a bronchus is brought close to surroundings of a lesion site. Then, an ultrasound probe to which a guide sheath is attached is inserted into a forceps channel formed in the bronchial endoscope. Furthermore, a distal end portion of the ultrasound probe is caused to protrude forward from a distal end of the bronchial endoscope to come close to the surroundings of the lesion site. Then, after a position or the like of the lesion site is perceived using the ultrasound probe, the ultrasound probe is removed from the forceps channel of the bronchial endoscope, and forceps as a biopsy instrument are instead newly inserted into the forceps channel. Moreover, a distal end portion of the forceps is caused to access the surroundings of the lesion site based on information about the perceived position, and living tissue in the lesion site is thereby sampled.

Now, U.S. Pat. No. 9,452,276 proposes a technique of performing biopsy using an endoscope and a biopsy needle. According to the technique, a predetermined endoscope is first inserted into a sheath that allows angle operation and that includes a shape measurement function for lumens. Then, the endoscope inserted into the sheath is inserted, together with the sheath, into a lumen of a subject, and a desired part is accessed using the shape measurement function of the sheath. Then, the endoscope is removed from the sheath, the biopsy needle is inserted into the sheath instead, and the biopsy needle is caused to access the desired part, and living tissue of the part is thus sampled.

There is also known a technique according to which an endoscope including a function of measuring a position of the endoscope is inserted, together with a sheath, into a lumen of a subject, and a desired lesion site is accessed using the position measurement function.

Now, it is sometimes difficult to cause an ultrasound probe to access a desired part such as a lesion using only information such as an optical image by an endoscope or a tomographic image by the ultrasound probe. In this regard, there is proposed a guide system for enabling a distal end portion of an ultrasound probe to easily reach a desired part.

More specifically, Japanese Patent Application Laid-Open Publication No. 2008-6108, Japanese Patent Application Laid-Open Publication No. 2010-69018, and the like each disclose a guide system that constructs three-dimensional data of an organ from a CT image that is acquired before observation or diagnosis of a subject, and that supports an insertion operation of an ultrasound probe by displaying an ultrasound observation position on the three-dimensional data.

### SUMMARY OF THE INVENTION

A living tissue sampling method according to a mode of the present invention includes: inserting an endoscope inserted into a guide sheath, into a body cavity of a subject, the guide sheath being a cylindrical member that is inserted into the body cavity of the subject, the guide sheath being configured to plastically deform by an internal component inserted inside the cylindrical member, the endoscope being configured to pick up an endoscopic image of inside of the body cavity of the subject, the endoscope being capable of outputting, to outside, distal end position information of the endoscope; bringing distal ends of the endoscope and the guide sheath close to a focused region at a time of insertion of the endoscope inserted into the guide sheath into the body cavity of the subject, by checking three-dimensional image information of a body cavity path inside the subject that is obtained by performing image pickup with respect to the subject by a device different from the endoscope, against distal end position data of the endoscope that is determined by acquiring distal end position information of the endoscope, and by associating a distal end position of the endoscope and a three-dimensional image with each other; then removing the endoscope from the guide sheath while keeping the guide sheath stationed inside the body cavity; then inserting a biopsy instrument into the guide sheath, and bringing the biopsy instrument inserted into the guide sheath close to the focused region; and then sampling living tissue of the focused region by the biopsy instrument.

Furthermore, a biopsy support system according to a mode of the present invention includes: a guide sheath configured as a cylindrical member that is inserted into a body cavity of a subject, the guide sheath being configured to plastically deform by an internal component inserted inside the cylindrical member; an endoscope that is insertable inside the cylindrical member of the guide sheath, the endoscope being configured to pick up an endoscopic image of inside of the body cavity of the subject and to be capable of outputting distal end position information of the endoscope to outside; an insertion support device configured to store three-dimensional image information of a body cavity path inside the subject that is obtained by performing image pickup with respect to the subject by a device different from the endoscope; a processor configured to determine distal end position data of the endoscope by acquiring the distal end position information of the endoscope, and to generate a navigation image showing a position, on a three-dimensional image, corresponding to a distal end position of the endoscope, based on the distal end position data of the endoscope; and a biopsy instrument that is insertable inside the cylindrical member of the guide sheath, the biopsy instrument being configured to sample living tissue of a focused region in the body cavity of the subject.

### BRIEF DESCRIPTION OF THE DRAWINGS

FIG. 1 is a block diagram showing a configuration of a biopsy support system according to an embodiment of the present invention;

FIG. 2 is an external perspective view showing an external appearance of a guide sheath of the biopsy support system of the embodiment, a state where an endoscope is inserted into the guide sheath, and a state where a biopsy instrument and an ultrasound probe are inserted into the guide sheath;

FIG. 3 is a flowchart showing a process, by an insertion support device of the biopsy support system of the embodiment, of acquiring CT image data, and generating and storing three-dimensional image information;

FIG. 4 is a flowchart showing a living tissue sampling process of the biopsy support system of the embodiment;

FIG. 5 is a diagram showing a state where the endoscope, of the biopsy support system of the embodiment, is brought close to a focused region in a bronchus in a state where the endoscope is inserted into the guide sheath; and

FIG. 6 is a diagram showing a state where the biopsy instrument and the ultrasound probe, of the biopsy support system of the embodiment, are brought close to the focused region in the bronchus in a state where the biopsy instrument and the ultrasound probe are inserted into the guide sheath.

### DETAILED DESCRIPTION OF THE PREFERRED EMBODIMENTS

Hereinafter, an embodiment of the present invention will be described with reference to the drawings.

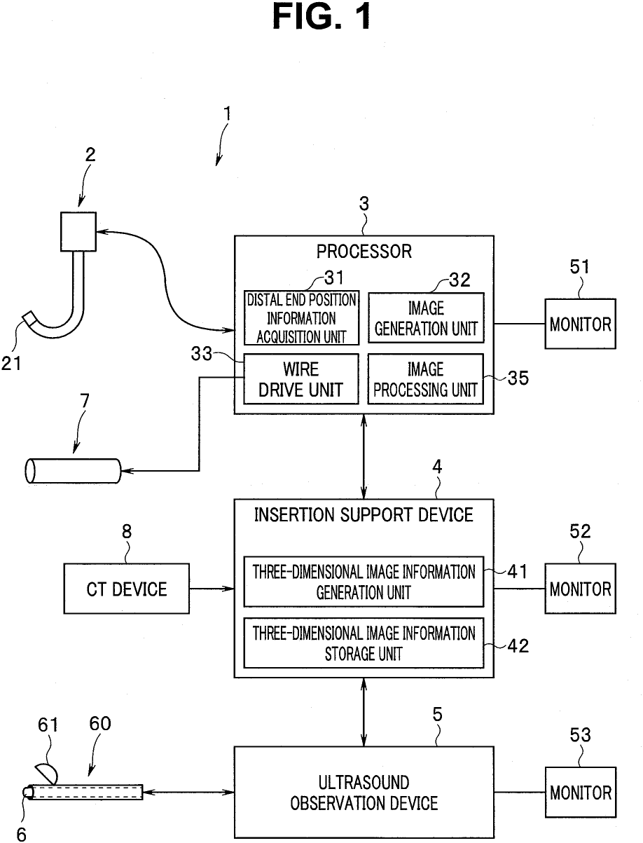

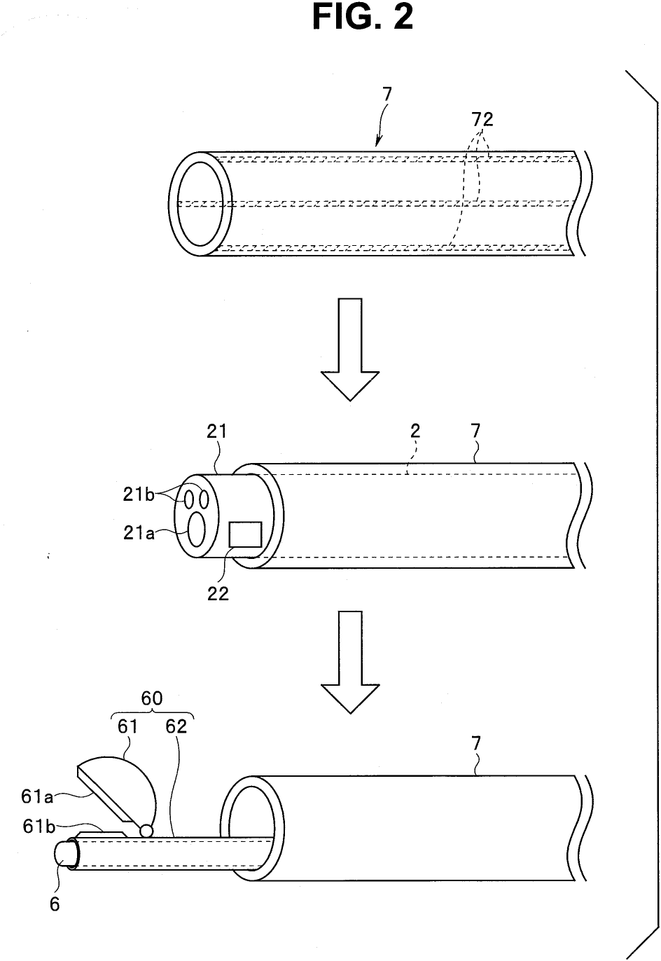

FIG. 1 is a block diagram showing a configuration of a biopsy support system according to an embodiment of the present invention, and FIG. 2 is an external perspective view showing an external appearance of a guide sheath of the biopsy support system of the embodiment, a state where an endoscope is inserted into the guide sheath, and a state where a biopsy instrument and an ultrasound probe are inserted into the guide sheath.

As shown in FIGS. 1 and 2, a biopsy support system 1 according to the embodiment of the present invention mainly includes an endoscope 2 that picks up an endoscopic image of inside of a body cavity of a subject, a processor 3 that is connected to the endoscope 2 and that outputs, to outside, the endoscopic image that is acquired after performing predetermined image processing, an insertion support device 4 that supports insertion of the endoscope 2 and an ultrasound probe 6, an ultrasound observation device 5 that controls ultrasound observation by the ultrasound probe 6, the ultrasound probe 6 that is connected to the ultrasound observation device 5 and that obtains a picked-up image of the inside of the body cavity of the subject, and a CT device 8 that acquires an X-ray tomographic image of the subject.

The biopsy support system 1 of the present embodiment further includes a guide sheath 7 that allows insertion of the endoscope 2 or the ultrasound probe 6 and that guides insertion into a lumen of a subject, and a biopsy instrument 60 for sampling living tissue of a lesion site in the lumen of the subject.

### Configuration of Guide Sheath 7

As shown in FIG. 2, in the present embodiment, the guide sheath 7 has a cylindrical shape that has an inner cavity portion. The guide sheath 7 allows insertion of an internal component such as the endoscope 2 or the ultrasound probe 6 into the inner cavity portion, and is plastically deformed by the internal component. The guide sheath 7 is inserted into a lumen of a subject with the internal component installed inside, while guiding the internal component.

Note that a sheath itself of the guide sheath 7 adopted in the present embodiment does not include a guide function, as will be described later, and thus, an outer diameter may be reduced and an inner diameter may be set to a relatively large diameter. More specifically, because the outer diameter may be made to have a dimension with which a periphery of the bronchus may be reached (for example, φ3.5 mm) and a sheath outer coat may be set to have a thin thickness, the inner diameter is set to approximately φ3.5 mm, which is a relatively large diameter for such a type of guide sheath.

Accordingly, that is, by adopting the guide sheath 7 having such dimensions, an endoscope that matches the large inner diameter can be applied and also relatively large biopsy instrument and ultrasound probe can be applied while allowing the periphery of the bronchus to be reached. For example, a biopsy forceps instrument that allows insertion of the ultrasound probe can be adopted.

Referring back to FIG. 2, as shown in a top part in FIG. 2, an angle adjustment wire 72 is disposed in the guide sheath 7, in a lengthwise direction of the cylindrical shape. The wire 72 is connected to a wire drive unit 33 in the processor 3, and an angle of the guide sheath 7 is changed by being controlled by the wire drive unit 33.

A middle part in FIG. 2 shows a state where the endoscope 2 is inserted into the inner cavity portion of the guide sheath 7, and where a distal end portion 21 of the endoscope 2 protrudes and is exposed from a distal end of the guide sheath 7.

Furthermore, a bottom part in FIG. 2 shows a state where the biopsy instrument 60 is inserted into the inner cavity portion of the guide sheath 7, instead of the endoscope 2, and where a distal end portion (a biopsy cup 61) of the biopsy instrument 60 protrudes and is exposed from the distal end of the guide sheath 7. Note that, in the present embodiment, the ultrasound probe 6 is inserted into an insertion hole in a main body portion 62 of the biopsy instrument 60, and FIG. 2 shows a state where a distal end portion of the ultrasound probe 6 protrudes from a main body inner cavity portion of the biopsy instrument 60.

Note that configurations of the endoscope 2, the ultrasound probe 6, and the biopsy instrument 60, and relationships between the members and the guide sheath 7 will be described later.

### Endoscopic System

The biopsy support system of the present embodiment includes an endoscopic system for observing and examining the bronchus of a subject, for example, and the endoscopic system of the present embodiment includes the endoscope (a bronchial endoscope) 2, the processor 3 to which the endoscope 2 is connected, the processor 3 being for controlling driving of the endoscope 2 and for performing predetermined image processing on a picked-up image from the endoscope 2, and a monitor 51 that outputs a video generated by the processor 3.

### Endoscope (Bronchial Endoscope) 2

As described above, in the present embodiment, the endoscope 2 is assumed to be a so-called bronchial endoscope. Note that the present invention is not limited to the biopsy support system including the bronchial endoscope, and is also applicable to a biopsy support system including other endoscopes such as an upper digestive tract endoscope, a large intestine endoscope, and the like.

The endoscope (the bronchial endoscope) 2 includes, inside a long and thin insertion portion, an image pickup optical system and an illumination optical system. The image pickup optical system includes an objective lens 21a disposed in the distal end portion 21 (see FIG. 2), and an image fiber and a relay lens, not shown, that are disposed on a proximal end side of the objective lens 21a. Picked-up light from a subject received by the objective lens 21a is inputted to an image pickup device that is disposed on the proximal end side through the image fiber and the relay lens.

An image pickup signal from the image pickup device that is disposed on a proximal end of the image pickup optical system is inputted to an image processing unit 35 of the processor 3, and is outputted to the monitor 51 after being subjected to image processing as appropriate.

Furthermore, the illumination optical system includes a light guide that transmits illumination light from a light source, not shown, and the illumination light is radiated from an illumination window 21b in the distal end portion 21.

Note that the endoscope (the bronchial endoscope) 2 of the present embodiment includes the image pickup optical system and the illumination optical system described above, but does not include a so-called forceps channel for allowing insertion of forceps and the like. Accordingly, forceps cannot be inserted into a lumen of a subject through the endoscope 2, but the endoscope is allowed to have a small diameter due to not including the forceps channel.

The endoscope 2 includes, at the distal end portion 21, a pattern light projection unit 22 that is a device that projects predetermined pattern light. The pattern light projection unit 22 includes a function of acquiring three-dimensional shape data of a lumen of a subject at a time of insertion of the endoscope 2 into the lumen, by projecting the predetermined pattern light toward the lumen.

Furthermore, the endoscope 2 projects the predetermined pattern light from the pattern light projection unit 22 under control of the processor 3 when inserted into the lumen of the subject, and transmits information that is obtained by projection (three-dimensional measurement information about the lumen that the endoscope 2 is facing), to a distal end position information acquisition unit 31 of the processor 3.

Note that with the processor 3, the distal end position information acquisition unit 31 acquires the endoscopic image that is obtained by projection of the pattern light and that is distal end position information, and determines distal end position data of the endoscope 2 based on the distal end position information.

Note that in the present embodiment, the three-dimensional shape data is determined by projecting the predetermined pattern light, but the three-dimensional shape data may alternatively be determined by other methods, such as by performing known stereo matching by including a stereo camera at the distal end portion 21, for example. The three-dimensional shape data may of course be determined by other known methods.

Furthermore, in the present embodiment, the distal end position data of the endoscope 2 inserted into a lumen of a subject is determined by the pattern light projection, but the distal end position data of the endoscope 2 may alternatively be determined by other components such as a sensor exemplified by an electromagnetic sensor or a shape sensor. However, the pattern light projection unit 22 that is adopted in the present embodiment can be made smaller than the electromagnetic sensor or the shape sensor, and thus, adopting detection of the distal end position data by the pattern light projection unit allows the endoscope to have a smaller diameter.

### Endoscope 2 and Guide Sheath 7

In the present embodiment, the endoscope 2 is inserted into a lumen of a subject, that is, the bronchus in the present embodiment, in a state of being inserted into the inner cavity portion of the guide sheath 7 having a cylindrical shape (see FIG. 2).

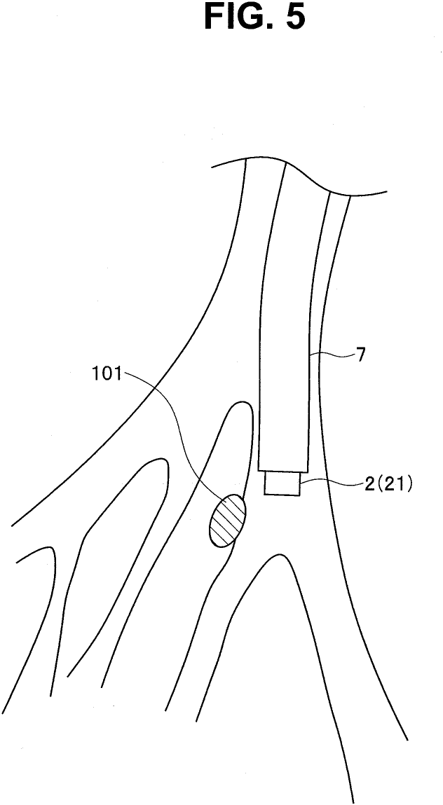

FIG. 5 is a diagram, according to the biopsy support system of the present embodiment, showing a state where the endoscope is brought close to a focused region in a bronchus in a state where the endoscope is inserted into the guide sheath.

As shown in FIG. 5, with the biopsy support system of the present embodiment, the bronchial endoscope 2 is first inserted into the inner cavity portion of the guide sheath 7 of the present embodiment having a relatively large diameter (see the middle part in FIG. 2), and the distal end portion of the guide sheath 7 and the distal end portion 21 of the endoscope 2 are brought close to a focused region (a lesion site 101) of the bronchus in this state. Note that the following process in the present embodiment will be described in detail later.

### Processor 3

The processor 3 allows connection of the endoscope 2, and controls driving of the image pickup device of the endoscope 2, and also, in the present embodiment, includes the distal end position information acquisition unit 31, an image generation unit 32, the wire drive unit 33, the image processing unit 35, and the like.

The image processing unit 35 includes a known image processing function, and acquires an image pickup signal from the image pickup device of the endoscope 2, performs predetermined image processing, and outputs a processing result to the monitor 51 as a video signal of the endoscopic image.

As described above, the distal end position information acquisition unit 31 acquires, from the endoscope 2, the endoscopic image that is obtained by projection of the pattern light and that is the distal end position information, and determines the distal end position data of the endoscope 2 based on the distal end position information.

The image generation unit 32 checks the distal end position data of the endoscope 2 that is obtained by the distal end position information acquisition unit 31 against three-dimensional image information that is stored in a three-dimensional image information storage unit 42 of the insertion support device 4, generates a navigation image showing a position, on a three-dimensional image, corresponding to a distal end position of the endoscope 2, and outputs the navigation image to a monitor 52.

Note that in the present embodiment, the processor 3 includes the distal end position information acquisition unit 31 and the image generation unit 32 described above, but one or both of the distal end position information acquisition unit 31 and the image generation unit 32 may be included in another device such as the insertion support device 4 described later, for example.

The wire drive unit 33 controls driving of the angle adjustment wire 72 (see FIG. 2) disposed in the guide sheath 7. The angle of the guide sheath 7 is controlled in up/down/left/right directions by the angle adjustment wire 72 being driven and controlled by the wire drive unit 33. Accordingly, an angle of the endoscope 2 or the ultrasound probe 6 that is inserted, as the internal component, into the guide sheath 7 is also controlled in a state where the endoscope 2 or the ultrasound probe 6 is inserted into the lumen of the subject. Note that the processor 3 may be a processor including a central processing unit (hereinafter referred to as a “CPU”), a ROM, a RAM, and the like, and various functions of the biopsy support system may be implemented by the CPU reading and executing software programs of various functions that are recorded in the ROM. With respect to the processor 3 here, the function of each unit may be implemented by individual hardware, or a part of each unit may be implemented by one piece of hardware, for example. For example, the processor 3 may include hardware, and the hardware may include at least one of a circuit that processes a digital signal or a circuit that processes an analog signal. Other than the central processing unit (CPU), the processor 3 may use various processors such as a DSP (digital signal processor). Moreover, the processor 3 may be an ASIC (application specific integrated circuit) or a hardware circuit based on an FPGA (field programmable gate array).

### Insertion Support System

The biopsy support system of the present embodiment includes an insertion support system, and the insertion support system includes a known CT device 8, the insertion support device 4 that acquires an X-ray tomographic image of a subject that is outputted from the known CT device 8, extracts a figure of a lumen of the subject, such as a bronchus, and constructs a virtual three-dimensional shape image, and that supports insertion of the endoscope 2 and the ultrasound probe 6, and the monitor 52 for combining and displaying a real image that is obtained from the endoscope 2 or the ultrasound probe 6 and a three-dimensional image of the subject.

### Insertion Support Device 4

In the present embodiment, the insertion support device 4 includes a CT image data acquisition unit, not shown, that acquires a CT image (an X-ray tomographic image) from the CT device 8, an image processing unit, not shown, that performs predetermined image processing on the CT image that is captured, a three-dimensional image information generation unit 41 that generates a predetermined three-dimensional image from the CT image that is captured, and a three-dimensional image information storage unit 42 that stores the three-dimensional image information.

The CT image data acquisition unit acquires the CT image (the X-ray tomographic image) from the CT device 8, and stores corresponding image data in a predetermined image data storage unit.

The image processing unit performs a process of extracting a bronchus based on the image data that is captured by the CT image data acquisition unit, by a process of extracting a body cavity part where air is present, for example.

The three-dimensional image information generation unit 41 includes a function of generating a three-dimensional image of the bronchus by a known method, based on the image data that is captured by the CT image data acquisition unit.

The three-dimensional image information storage unit 42 stores the three-dimensional image information of the bronchus generated by the three-dimensional image information generation unit 41.

The three-dimensional image information of the bronchus of the subject stored by the three-dimensional image information storage unit 42 is transmitted to the processor 3 and the ultrasound observation device 5, and the three-dimensional image information contributes to generation of the navigation image that shows the position, on the three-dimensional image, corresponding to the distal end position of the endoscope 2 or the ultrasound probe 6.

The CT device 8 is a known CT (computed tomography) device that picks up an X-ray tomographic image of a subject, and in the biopsy support system of the present embodiment, the X-ray tomographic image of a subject is picked up and is transmitted to the insertion support device 4 in advance before observation, diagnosis, and biopsy of the subject.

### Ultasound Obervation System

The biopsy support system of the present embodiment includes an ultrasound observation system, and the ultrasound observation system includes the ultrasound probe 6, the ultrasound observation device 5 to which the ultrasound probe 6 is connected and that includes an ultrasound image generation unit for generating an ultrasound tomographic image, and a monitor 53 that displays the ultrasound tomographic image generated by the ultrasound observation device 5.

### Ultrasound Probe 6

The ultrasound probe 6 includes a known ultrasound transducer, and acquires an ultrasound image based on a reflected wave received from the subject, and outputs the ultrasound image to the ultrasound observation device 5.

Furthermore, the ultrasound probe 6 includes a thin and long probe main body and a probe distal end portion, and the ultrasound transducer is disposed in the probe distal end portion.

The ultrasound transducer is driven based on a drive signal from the ultrasound observation device 5 to emit an ultrasound beam inside a living body. Furthermore, the ultrasound transducer receives a reflected wave that is the ultrasound beam that is reflected by living tissue inside the living body, and outputs an echo signal according to the reflected wave to the ultrasound observation device 5.

Note that the ultrasound observation system may include, together with the ultrasound probe 6, a position detection device, not shown, for detecting the distal end position of the ultrasound probe 6. In this case, the ultrasound probe 6 includes, at the distal end portion, a known position sensor for detecting a position of the ultrasound transducer, and the position detection device detects a three-dimensional position of the position sensor mentioned above of the ultrasound probe 6. Note that a position detection method of the position detection device and the ultrasound probe 6 including the position sensor mentioned above uses a known technique, and a detailed description is omitted.

### Biopsy Instrument 60

The present embodiment adopts, as a biopsy instrument for sampling living tissue of a lesion site in a lumen (a bronchus) of a subject, the biopsy instrument 60 including inside an insertion hole that allows insertion of the ultrasound probe 6.

As shown in FIG. 2, the biopsy instrument 60 includes the main body portion 62 that is a long cylindrical portion, and the biopsy cup 61 that is disposed at a distal end portion of the main body portion 62. The biopsy cup 61 is displaceable by a drive mechanism, not shown, and includes a grasping portion 61a for sampling living tissue. A grasping portion 61b for grasping the living tissue together with the grasping portion 61a is formed at a part, of the distal end portion of the main body portion 62, facing the grasping portion 61a.

The biopsy cup 61 of the present embodiment includes a function of sampling a large amount of living tissue of a lesion site at the time of performing sampling, compared to a regular biopsy cup. This is because a cup portion with a relatively large capacity can be adopted thanks to the inner diameter of the guide sheath 7 adopted in the present embodiment being relatively large, as will be described later.

Note that a shape of the biopsy cup 61 and configurations of the grasping portions 61a, 61b are not limited to the shape and the configurations described above, and various modes including a living tissue sampling function may be adopted.

Furthermore, the biopsy instrument 60, including the biopsy cup 61 described above, is set to a dimension that can be inserted into the inner cavity portion of the guide sheath 7. In other words, as described above, the inner diameter of the guide sheath 7 of the present embodiment is approximately φ3.5 mm, and thus, a relatively large biopsy instrument can be applied.

Moreover, as described above, the main body portion 62 of the biopsy instrument 60 includes the insertion hole that allows insertion of the ultrasound probe 6. In other words, in the present embodiment, the biopsy instrument 60 can be entirely, including the biopsy cup 61, inserted into the inner cavity portion of the guide sheath 7 in a state where the ultrasound probe 6 is inserted into the insertion hole in the main body portion 62 (see the bottom part in FIG. 2).

In other words, because sampling of living tissue by the biopsy instrument 60 can be performed while implementing the function of the ultrasound probe 6, biopsy by so-called real-time sampling can be performed.

What is important here is that there are advantageous effects that, as described above, the guide sheath 7 of the present embodiment has an outer diameter that is set to a dimension by which the periphery of a bronchus can be reached (for example, φ3.5 mm) and an inner diameter that is set to φ3.5 mm that is approximately the same as the outer diameter thanks to a reduced thickness of the sheath outer coat due to not including the guide function, and is thus able to reach the periphery, and also, the biopsy cup 61 having a large capacity, as in the case of the biopsy instrument 60, can be adopted, and moreover, the real-time sampling function using the ultrasound probe 6 can be achieved.

### Ultrasound Probe 6, Bopsy Instrument 60, and Guide Sheath 7

As described above, in the present embodiment, the ultrasound probe 6 is inserted into the lumen of a subject in a state of being inserted into an inner cavity of the guide sheath 7 (see FIG. 2).

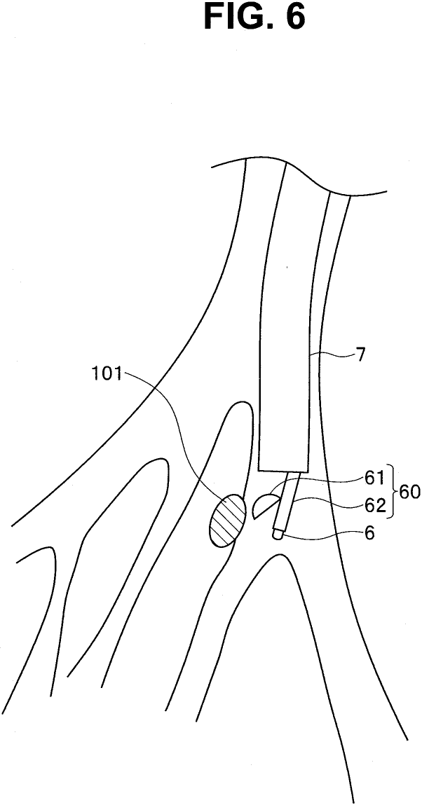

FIG. 6 is a diagram showing a state where the biopsy instrument and the ultrasound probe, of the biopsy support system of the present embodiment, are brought close to a focused region in a bronchus in a state where the biopsy instrument and the ultrasound probe are inserted into the guide sheath.

As shown in FIG. 6, with the biopsy support system of the present embodiment, the endoscope 2 inserted into the guide sheath 7 is first brought close to a focused region (the lesion site 101) of the bronchus, and then, the endoscope 2 is removed in a state where the guide sheath 7 is kept stationed and the biopsy instrument 60 is inserted into the guide sheath 7 instead.

Furthermore, the ultrasound probe 6 is inserted into the insertion hole in the main body portion 62 of the biopsy instrument 60 and the biopsy cup 61 of the biopsy instrument 60 is brought close to the lesion site 101, and the distal end portion of the ultrasound probe 6 is also brought close. Then, the real-time sampling function is implemented while operating the ultrasound probe 6, and living tissue in the lesion site 101 is sampled by the biopsy cup 61.

### Ultrasound Observation Device 5

In the present embodiment, the ultrasound observation device 5 allows connection of the ultrasound probe 6, and includes a known ultrasound observation function. In other words, the ultrasound observation device 5 generates and outputs a drive signal for driving the ultrasound transducer of the ultrasound probe 6, and generates an ultrasound image as a tomographic image of a living body successively at a predetermined frame rate, based on an echo signal from the ultrasound transducer of the ultrasound probe 6. Furthermore, the ultrasound observation device 5 successively outputs the ultrasound image that is generated, to the processor 3.

Note that the ultrasound tomographic image generated by the ultrasound observation device 5 is displayed on the monitor 53, and other workings and effects are the same as the workings and effects of a known ultrasound observation device, and a detailed description will be omitted.

### Working of Present Embodiment

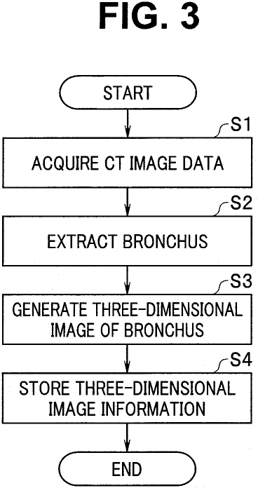

Next, a process of the biopsy support system of the present embodiment, of generating and storing the three-dimensional image information will be described with reference to FIG. 3.

FIG. 3 is a flowchart showing a working of a process by the insertion support device of the biopsy support system of the embodiment, of acquiring CT image data, and generating and storing the three-dimensional image information.

With the biopsy support system of the present embodiment, before sampling of living tissue of a lesion site in a lumen (a bronchus) of a subject, the insertion support device 4 acquires the CT image data from the CT device 8, and generates and stores the three-dimensional image information based on the CT image data.

In other words, as shown in FIG. 3, the CT image data acquisition unit of the insertion support device 4 first acquires the CT image (the X-ray tomographic image) from the CT device 8 (step S1). Then, the image processing unit of the insertion support device 4 performs a process of extracting a bronchus based on the image data that is captured by the CT image data acquisition unit by, for example, a process of extracting a body cavity part where air is present (step S2).

Next, the three-dimensional image information generation unit 41 of the insertion support device 4 generates the three-dimensional image of the bronchus by a known method based on the image data that is captured by the CT image data acquisition unit (step S3).

Furthermore, the three-dimensional image information storage unit 42 of the insertion support device 4 stores the three-dimensional image information of the bronchus that is generated by the three-dimensional image information generation unit 41 (step S4).

Note that the three-dimensional image information of the bronchus of the subject stored in the three-dimensional image information storage unit 42 is transmitted to the processor 3 and the ultrasound observation device 5, and the three-dimensional image information contributes to generation of the navigation image that shows the position, on the three-dimensional image, corresponding to the distal end position of the endoscope 2 or the ultrasound probe 6.

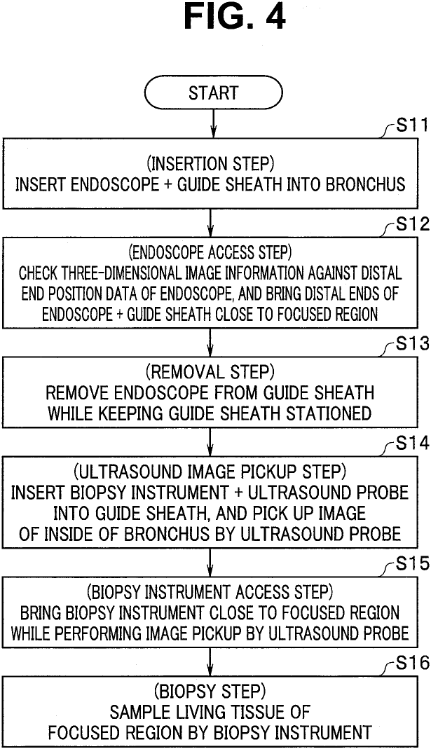

Next, a living tissue sampling process of the biopsy support system of the present embodiment will be described with reference to FIG. 4.

FIG. 4 is a flowchart showing the living tissue sampling process of the biopsy support system of the embodiment.

As shown in FIG. 4, at the time of sampling living tissue of a lesion site in a lumen (a bronchus) of a subject, a surgeon first inserts the endoscope 2 into the inner cavity portion of the guide sheath 7 having a cylindrical shape, and inserts the endoscope 2 and the guide sheath 7, in this state (see the middle part in FIG. 2), into the lumen of the subject, that is, the bronchus in the present embodiment (step S11; insertion step).

Next, in the state where the bronchial endoscope 2 is inserted into the inner cavity portion of the guide sheath 7, the surgeon brings the distal end portion of the guide sheath 7 and the distal end portion 21 of the endoscope 2 close to a focused region (the lesion site 101) of the bronchus (step S12; endoscope access step).

A working of the endoscope 2, the processor 3, and the insertion support device 4 in the endoscope access step will now be described.

First, the endoscope 2 projects the predetermined pattern light from the pattern light projection unit 22 under control of the processor 3, and transmits information that is obtained by projection (the distal end position information that is an endoscopic image that is the three-dimensional measurement information of the lumen that the endoscope 2 is facing) to the distal end position information acquisition unit 31 of the processor 3.

The distal end position information acquisition unit 31 of the processor 3 acquires, from the endoscope 2, the endoscopic image that is obtained by projection of the pattern light and that is the distal end position information, and determines the distal end position data of the endoscope 2 based on the distal end position information.

Furthermore, the image generation unit 32 of the processor 3 checks the distal end position data of the endoscope 2 that is obtained by the distal end position information acquisition unit 31 against the three-dimensional image information that is stored in the three-dimensional image information storage unit 42 of the insertion support device 4, generates the navigation image showing a position, on the three-dimensional image, corresponding to the distal end position of the endoscope 2, and outputs the navigation image to the monitor 52.

In the endoscope access step, the surgeon brings the distal end portion of the guide sheath 7 and the distal end portion 21 of the endoscope 2 close to the focused region (the lesion site 101) of the bronchus, based on the navigation image (see FIG. 5).

Next, in a stage where the endoscope 2 and the guide sheath 7 are brought close to the lesion site 101, the surgeon removes the endoscope 2 from the guide sheath 7 while keeping the guide sheath 7 stationed at the position (step S13; removal step).

Then, the surgeon inserts the biopsy instrument 60 and the ultrasound probe 6 into the guide sheath 7 that is stationed. In other words, the biopsy instrument 60 and the ultrasound probe 6 are inserted into the inner cavity portion of the guide sheath 7 that is kept stationed near the lesion site 101, in a state where the ultrasound probe 6 is inserted into the insertion hole of the main body portion 62 of the biopsy instrument 60.

Then, in a stage where the biopsy instrument 60 and the distal end portion of the ultrasound probe 6 slightly protrude from the distal end of the guide sheath 7, image pickup is performed by the ultrasound probe 6 in relation to surroundings of the lesion site (step S14; ultrasound image pickup step).

Moreover, the surgeon brings the biopsy cup 61 of the biopsy instrument 60 close to the lesion site 101 that is the focused region, based on image pickup information from the ultrasound probe 6 (step S15; biopsy instrument access step, see FIG. 6).

Then, the surgeon performs a biopsy process by sampling living tissue of the lesion site by the biopsy instrument 60 (step S16; biopsy step).

As described above, the biopsy support system of the present embodiment includes the guide sheath 7 that has an outer diameter that is set to a dimension by which a periphery of a bronchus can be reached (for example, φ3.5 mm), and an inner diameter that is set to φ3.5 mm that is approximately the same as the outer diameter thanks to a reduced thickness of the sheath outer coat due to not including the guide function. Furthermore, the biopsy support system of the present embodiment achieves, by adopting the guide sheath 7 as described above, excellent advantageous effects that the biopsy cup 61 having a large capacity, as in the case of the biopsy instrument 60, can be adopted, and a real-time sampling function using the ultrasound probe 6 can be achieved, while allowing the periphery to be reached.

Furthermore, the biopsy support system of the present embodiment determines the distal end position data by pattern light projection by the pattern light projection unit 22 of the endoscope 2 at the time of insertion, into a lumen of a subject, of the endoscope 2 held inside the guide sheath 7, and there is an advantageous effect that the diameter of the endoscope can be more reduced than in the case of determining the distal end position data by other methods by using an electromagnetic sensor or a shape sensor, for example.

As described above, the biopsy support system of the present embodiment achieves advantageous effects that a desired part of a lumen of a subject can be accurately accessed, and that sheath outer diameter and inner diameter suitable for peripheral procedures, that is, a reduction in the outer diameter and an increase in the inner diameter, can both be achieved.

The present invention is not limited to the embodiment described above, and various changes and modifications may be made within the gist of the present invention.

What is claimed is:

1. A living tissue sampling method comprising:
inserting an endoscope inserted into a guide sheath, into a body cavity of a subject, the guide sheath being a cylindrical member that is inserted into the body cavity of the subject, the guide sheath being configured to plastically deform by an internal component inserted inside the cylindrical member, the endoscope being configured to pick up an endoscopic image of inside of the body cavity of the subject, the endoscope being capable of outputting, to outside, distal end position information of the endoscope;
bringing distal ends of the endoscope and the guide sheath close to a focused region at a time of insertion of the endoscope inserted into the guide sheath into the body cavity of the subject, by checking three-dimensional image information of a body cavity path inside the subject that is obtained by performing image pickup with respect to the subject by a device different from the endoscope, against distal end position data of the endoscope that is determined by acquiring distal end position information of the endoscope, and by associating a distal end position of the endoscope and a three-dimensional image with each other;
then removing the endoscope from the guide sheath while keeping the guide sheath stationed inside the body cavity;
then inserting a biopsy instrument into the guide sheath, and bringing the biopsy instrument inserted into the guide sheath close to the focused region; and
then sampling living tissue of the focused region by the biopsy instrument.

2. The living tissue sampling method according to claim 1, wherein an image pickup appliance with an outer diameter smaller than an outer diameter of the endoscope is inserted into the guide sheath, and the image pickup appliance picks up an image of the body cavity path in the subject.

3. The living tissue sampling method according to claim 2, wherein the biopsy instrument that is inserted into the guide sheath is brought close to the focused region while referring to the image of the body cavity path picked up by the image pickup appliance.

4. The living tissue sampling method according to claim 2, wherein when the distal ends of the endoscope and the guide sheath are brought close to the focused region,
the endoscope acquires the distal end position information of the endoscope based on three-dimensional shape data,
a processor that is connected to the endoscope determines the distal end position data of the endoscope from the distal end position information that is the three-dimensional shape data, and
the processor checks the distal end position data of the endoscope that is obtained against the three-dimensional image information, and generates a navigation image showing a position, on the three-dimensional image, corresponding to a distal end position of the endoscope.

5. The living tissue sampling method according to claim 4, wherein when the distal ends of the endoscope and the guide sheath are brought close to the focused region,
the endoscope including, at a distal end portion, a pattern light projection device configured to project pattern light acquires the distal end position information of the endoscope based on the three-dimensional shape data by acquiring an endoscopic image that is obtained by projection of the pattern light, and
the processor that is connected to the endoscope determines the distal end position data of the endoscope from the distal end position information by acquiring the endoscopic image that is obtained by projection of the pattern light and that is the distal end position information.

6. A biopsy support system comprising:
a guide sheath configured as a cylindrical member that is inserted into a body cavity of a subject, the guide sheath being configured to plastically deform by an internal component inserted inside the cylindrical member;
an endoscope that is insertable inside the cylindrical member of the guide sheath, the endoscope being configured to pick up an endoscopic image of inside of the body cavity of the subject and to be capable of outputting distal end position information of the endoscope to outside;
an insertion support device configured to store three-dimensional image information of a body cavity path inside the subject that is obtained by performing image pickup with respect to the subject by a device different from the endoscope;
a processor configured to determine distal end position data of the endoscope by acquiring the distal end position information of the endoscope, and to generate a navigation image showing a position, on a three-dimensional image, corresponding to a distal end position of the endoscope, based on the distal end position data of the endoscope; and
a biopsy instrument that is insertable inside the cylindrical member of the guide sheath, the biopsy instrument being configured to sample living tissue of a focused region in the body cavity of the subject.

7. The biopsy support system according to claim 6, further comprising an image pickup appliance that is insertable inside the cylindrical member of the guide sheath, the image pickup appliance being configured to pick up an image of inside of the body cavity of the subject.

8. The biopsy support system according to claim 7, wherein the biopsy instrument includes an insertion hole for allowing insertion of the image pickup appliance.

9. The biopsy support system according to claim 8, wherein
the image pickup appliance is an ultrasound probe configured to emit an ultrasonic wave toward the subject and to acquire an ultrasound image based on a reflected wave from the subject, and
the ultrasound probe is inserted into the insertion hole of the biopsy instrument.

10. The biopsy support system according to claim 6, wherein the biopsy instrument is forceps.

11. The biopsy support system according to claim 6, wherein
the endoscope acquires the distal end position information of the endoscope based on three-dimensional shape data,
the processor determines the distal end position data of the endoscope from the distal end position information that is the three-dimensional shape data, and
the processor checks the distal end position data of the endoscope that is obtained against the three-dimensional image information that is stored, and generates a navigation image showing a position, on the three-dimensional image, corresponding to the distal end position of the endoscope.

12. The biopsy support system according to claim 11, wherein
the endoscope includes, at a distal end portion, a pattern light projection device configured to project pattern light, and acquires the distal end position information of the endoscope based on the three-dimensional shape data by acquiring an endoscopic image that is obtained by projection of the pattern light, and
the processor that is connected to the endoscope determines the distal end position data of the endoscope from the distal end position information by acquiring the endoscopic image that is obtained by projection of the pattern light and that is the distal end position information.

13. The biopsy support system according to claim 6, wherein the three-dimensional image information is acquired by an X-ray CT device.

14. The biopsy support system according to claim 6, wherein the body cavity of the subject belongs to a respiratory system.
